(19) United States
(12) Patent Application Publication
(10) Pub. No.: US 2016/0151141 A1
(43) Pub. Date: Jun. 2, 2016

(54) High Flow Embolic Protection Device

(71) Applicant: LAKE REGION MANUFACTURING, INC., Chaska, MN (US)

(72) Inventor: John Mathew Zimmerman, Minnetrista, MN (US)

(21) Appl. No.: 14/905,933

(22) PCT Filed: Jul. 15, 2014
(86) PCT No.: PCT/US2014/046591

§ 371 Date: Jan. 18, 2016

Related U.S. Application Data
(60) Provisional application No. 61/847,209, filed on Jul. 17, 2013.

(51) Int. Cl. A61F 2/01; A61F 2/89

(52) U.S. Cl. CPC A61F 2/01; A61F 2/89; A61F 2002/016; A61F 2002/011; A61F 2230/0069

(57) ABSTRACT

The present invention relates to apparatus and methods tor providing embolic protection in a patient's vascular system, in particular, it relates to an embolic protection device comprising, in one embodiment, braided mesh-like tubular embolus collection structure or structures that can be deployed in a patient's aorta to protect the aortic arch vessels and downstream organs from, embolus formation by collecting and removing emboli from the blood stream. This embolic protection device can be used acutely, for example for embolic protection during cardiac surgery and interventional cardiology procedures, or it can be implanted for chronic embolic protection, for example from cardiogenic emboli or emboli front ruptured or vulnerable aortic plaque. In one coaxial embodiment, multiple i.e., 20 or more, braided, mesh-like tubular structures are used. In a farther embodiment multiple coaxial braided mesh-like tubular structures are used having varying degrees of porosity, the inner-most tubular structure being most porous and the outer-most tubular structure being the last porous.

### FIELD OF THE INVENTION

The present invention relates to apparatus and methods for providing embolic protection in a patient's vascular system. In particular, it relates to an embolic protection device that can be deployed in a patient's aorta to protect the aortic arch vessels and downstream organs from potential emboli. The embolic protection device can be used acutely, for example for embolic protection during cardiac surgery and interventional cardiology procedures, or it can be implanted for chronic embolic protection, for example from cardiogenic emboli or emboli from ruptured or vulnerable aortic plaque.

Cerebral embolism is a known complication of cardiac surgery, cardiopulmonary bypass and catheter-based interventional cardiology and electrophysiology procedures such as, but not limited to, transcatheter aortic valve replacement implantation TAVR/TAVI. Embolic particles, which may include thrombus, atheroma and lipids, may become dislodged by surgical or catheter manipulations and enter the bloodstream, embolizing in the brain or other vital organs downstream. Other sources of potential emboli include cardiogenic emboli, such as thrombus that results from chronic atrial fibrillation, and emboli from ruptured or vulnerable aortic plaque. Cerebral embolism can lead to neuropsychological deficits, stroke and even death. Other organs downstream can also be damaged by embolism, resulting in diminished function or organ failure. Prevention of embolism formation by capture or collection of antegrade-flowing embolic debris benefits patients and substantially improves the outcome of the various procedures with which it is used.

Given that the sources of potential emboli can be acute or chronic, it would be advantageous to provide an embolic protection device that can either be used acutely, for example for embolic protection during cardiac surgery and interventional cardiology procedures, or that can be implanted for chronic embolic protection, for example from cardiogenic emboli or emboli from ruptured or vulnerable aortic plaque. A further advantage would be realized by providing an embolic protection device that can be implanted without interfering with transluminal aortic access for performing future surgeries and other interventional or diagnostic procedures. Another advantage would come from providing an embolic protection device that can be retrieved and removed from the patient after the necessity for it has passed. Yet another advantage would come from providing an embolic protection device that can be deployed and retrieved using minimally invasive techniques.

The embolic protection device of this application is characterized as being “High Flow.” By this it is meant that the device of this application is particularly adapted to capture emboli in vascular or aortic locations where larger blood volumes or higher blood pressure or both is found. For example, a preferred location for deployment of this protection device within or adjacent to the aortic arch. In such high flow locations this device can filter emboli from large volumes of blood with minimal creation of back flow or back pressure. Back pressure or back flow gradients as are sometime created by emboli protection devices are generally to be avoided so as not to cause the heart to work harder to produce the required cardiac output.

Previous devices for preventing cerebral embolism are described in the following patents and patent applications, which are hereby incorporated by reference: U.S. Pat. App. 20040215167 Embolic Protection Device, PCT App. WO/2004/019817 Embolic Protection Device, U.S. Pat. No. 6,371,935 Aortic Catheter with Flow Divider and Methods for Preventing Cerebral Embolization, U.S. Pat. No. 6,361,545 Perfusion Filter Catheter, U.S. Pat. No. 6,254,563 Perfusion Shunt Apparatus and Method, U.S. Pat. No. 6,139,517 Perfusion Shunt Apparatus and Method, U.S. Pat. No. 6,537,297 Methods of Protecting a Patient from Embolization during Surgery, U.S. Pat. No. 6,499,487 Implantable Cerebral Protection Device and Methods of Use, U.S. Pat. No. 5,769,816 Cannula with Associated Filter, U.S. Pat. App. 20030100940 Implantable Intraluminal Protector Device and Method of Using Same for Stabilizing Atheromas.

### BRIEF DESCRIPTION OF THE DRAWINGS

It is to be understood that the drawings and the description below are provided primarily for purposes of facilitating understanding the conceptual aspects of the invention and various possible embodiments thereof, including what is presently considered to be preferred embodiments. It is to be further understood that the embodiments described are for purposes of example only, and that the invention is capable of being embodied in other forms and applications than described herein as will be suggested to one skilled in this art in view of the present disclosure, figures, and claims.

FIG. 1 shows basic coronary anatomy discussed with respect to this invention.

FIG. 2 shows an embolic protection device of this invention in its implanted, expanded condition.

FIG. 3 shows the embolic protection device of FIG. 2 in an undeployed or retracted condition.

FIG. 4 shows an embolic protection device in an undeployed condition being inserted into a patient's aortic arch.

FIG. 5 shows an embolic protection device in a retracted condition for removal from the patient's aorta.

FIG. 6 shows schematically an embodiment of the present embolic protection device 100 partially deployed into the left subclavian artery 300.

FIG. 7 shows the embodiment of FIG. 6 in which a TAVR catheter 302 accesses the aortic valve through the present embolic device.

FIG. 8 shows deployment of a device of this invention 100′ into the descending aorta. The arrows 200 show possible directions of blood flow after emboli have been captured or filtered therefrom by the present device.

FIG. 9 shows the location in the descending aorta where emboli would be captured by the device shown in FIG. 8.

FIG. 10 illustrates withdrawal of device of this invention containing captured emboli using a large lumen catheter.

FIG. 11 shows the device of FIG. 10 with attachment points asserting embolic protection device withdrawal.

FIG. 12 shows capture of the emboli-containing device by withdrawal of same into the catheter.

FIG. 13 illustrates a recapture hook 202 which can be used to assist device retrieval and withdrawal.

FIG. 14 illustrates and describes a further approach to embolic protection device withdrawal.

FIG. 15 is a detailed depiction of emboli being captured by an embolic protection device of the invention along the wall of the aorta.

FIG. 16 shows an embodiment of the invention in which a porous membrane of polymer sieve or membrane 204 is deployed within, upon or outside the embolic protection device to capture emboli.

FIG. 17A shows a detailed schematic sectioned representation of emboli capture using a triaxial protection device of this invention.

FIGS. 17B and 17C show a side and perspective view of the triaxial braided mesh embodiment of this embolic protection device.

FIGS. 17D and 17E show partially assembled exploded views of the device shown in FIGS. 17A through 17C.

FIGS. 18a and 18b are side and end view, respectively, illustrating one form of intraluminal device constructed in accordance with the present invention, the device being shown in its implanted, expanded condition.

FIGS. 19a and 19b are corresponding views but illustrating the device in its contracted, stressed condition.

FIG. 20 more particularly illustrates the braid pattern of FIGS. 18a, 18b and 19a, 19b in the expanded condition of the braided tube.

FIG. 21 illustrates another braid pattern, wherein one filament extending in one helical direction is interwoven over and under two filaments extending in the opposite helical direction.

FIG. 22 illustrates a further braid pattern in which two (or more) contiguous filaments extending helically in one direction are interwoven over and under two (or more) contiguous filaments extending in the opposite direction.

FIG. 23 schematically shows the relationship between the bending rigidity of the braided tube with respect to the diameter of the filaments producing the braided tube.

### BRIEF SUMMARY OF THE INVENTION

The present invention, in one aspect, provides a high-flow intraluminal embolic protection device implantable in a blood vessel, the device comprising: a braided mesh-like tube of bio-compatible material having an expanded condition in which the tube diameter is larger than the diameter of the blood vessel in which it is to be implanted, the braided mesh-like tube having a length sufficient to be anchored to the source blood vessel, the braided mesh-like tube being dimensioned and configured to have in its implanted condition a porosity index such as to filter or capture antegrade-flowing emboli but not to unduly reduce the blood flow.

### DETAILED DESCRIPTION OF THE INVENTION

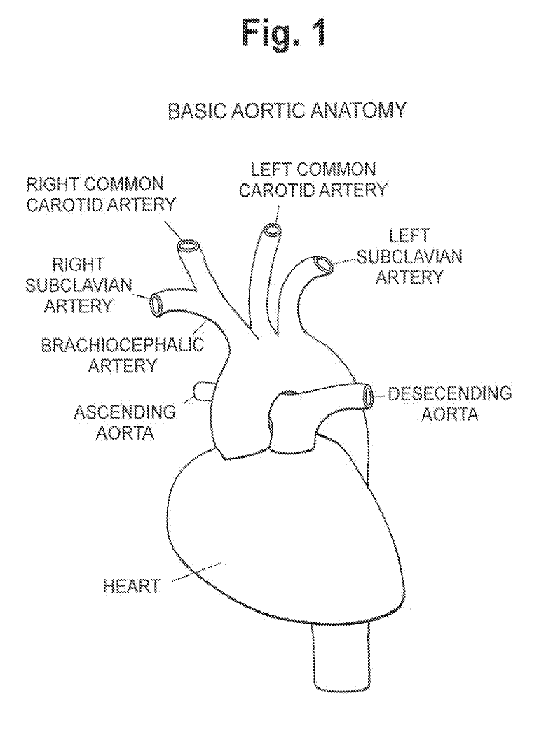

FIG. 1 shows schematically basic aortic anatomy relevant to one aspect of this invention i.e., when the present embolic protection device or filter is employed in conjunction with a TAVI or TAVR procedure. It is to be understood that the present invention can be deployed before, during, or after a transcatheter procedure in which emboli may be generated. Oxygenated blood flows from the heart to the ascending aorta, to the arch of aorta to the right and left subclavian arteries, the right and left carotid arteries, and to the descending aorta. FIG. 1 is used schematically, generally in section, to illustrate the features of this invention in several of the FIGs which follow.

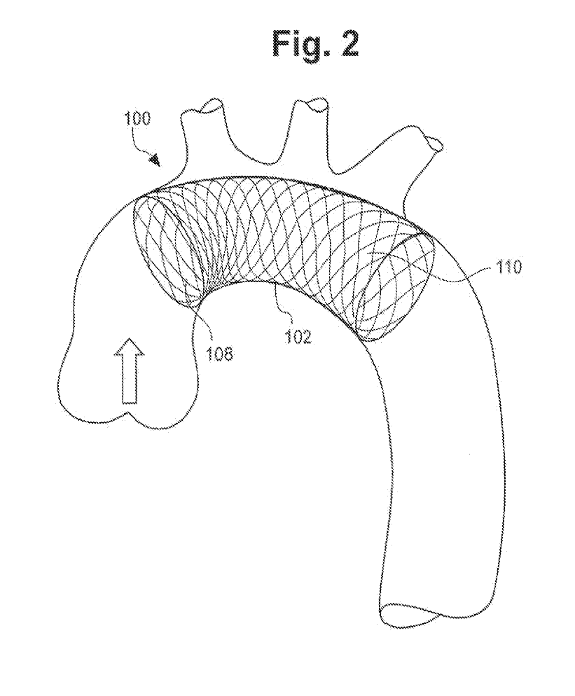

FIG. 2 shows an embolic protection device 100 according to the present invention in an expanded or deployed condition. It will be recognized that device 102 is deployed within the aortic arch. The embolic protection device 100 has an approximately cylindrical outer structure 102 made of a braided mesh-like material. Device 100 has an upstream end 108 and downstream end 110. The upstream end 108 of the embolic protection device 100 is open for blood to flow as indicated by the arrow in FIG. 2. The braided mesh-like material (sometimes referred to as “filter mesh material”) of the cylindrical outer structure 102 may be made of knitted, woven or nonwoven fibers, filaments or wires and will have a pore size chosen to prevent emboli above a certain size from passing through. The filter mesh material may be made of a metal, a polymer or a combination thereof and may optionally have an antithrombogenic coating on its surface. The filter mesh material of the cylindrical outer structure 102 may have a pore size in the range of approximately 1 mm to 0.1 mm or even smaller, depending on whether it is intended to capture macroemboli only or microemboli as well. Alternatively, the filter mesh material of the cylindrical outer structure 102 may have a pore size to stop microemboli as small as 0.1 mm.

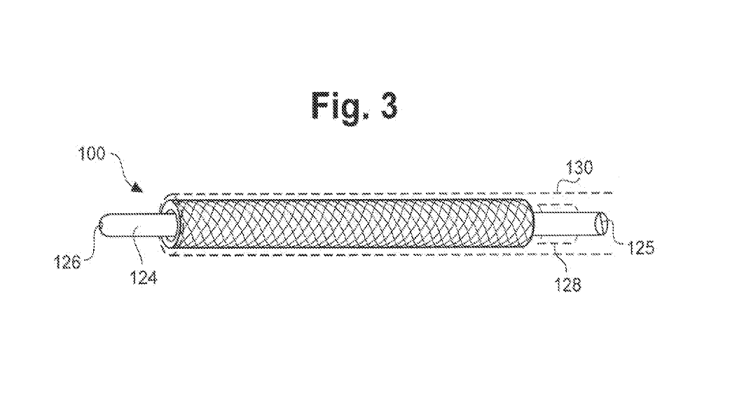

FIG. 3 shows the embolic protection device 100 of FIG. 1 in an undeployed or retracted condition. Typically, delivery catheter 124 will be used, the delivery catheter 124 constructed with an internal lumen 125 that terminates in a guidewire port 126 at the distal end of the catheter 124. Optionally, a tubular outer delivery sheath 130 shown in dashed line may be used to maintain the embolic protection device 100 in the undeployed condition. The delivery catheter 124 may optionally include a shoulder or other retention structure 128 positioned proximal to the embolic protection device 100 to maintain the position of the embolic protection device 100 on the delivery catheter 124 as the delivery sheath 130 is withdrawn during deployment. Alternatively, a pusher catheter (not shown) that fits in between the delivery catheter 124 and the delivery sheath 130 may be used to facilitate deployment.

Optionally, when the embolic protection device 100 is intended to be used for embolic protection during a catheter-based diagnostic or interventional procedure, the delivery catheter 124 may be configured as a diagnostic catheter, a guiding catheter, or therapeutic catheter.

The embolic protection device 100 will preferably be self-supporting in the deployed condition. This can be accomplished with a variety of different constructions. In one example, the cylindrical outer structure 102 can be constructed with a resilient filter mesh material that can be compressed into the undeployed condition and will self-expand into the deployed condition. The filter mesh can be resilient, flaccid or plastically deformable.

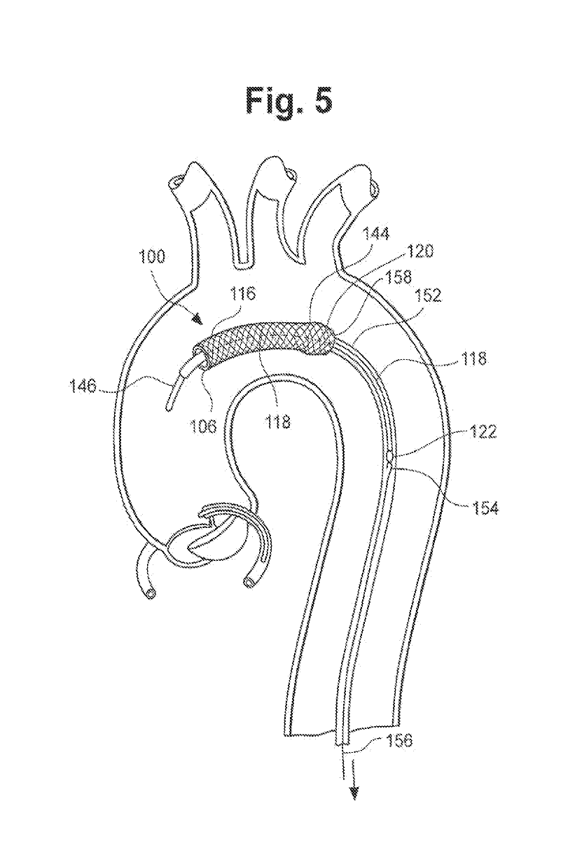

Hybrid constructions that combine features of the self-supporting structure and the frame-supported structure. Hybrid deployment methods, such as balloon-assisted self-expansion can also be utilized. Optionally, the embolic protection device 100 may include features to assist in retracting the device for retrieval from the patient's aorta (See, FIG. 5 discussion below). For example, the upstream end 108 and the downstream end 110 of the embolic protection device 100 may be constructed with retraction members 116, 120 that are configured like purse strings or lassos around the circumference of the cylindrical outer structure 102. A pull loop 122 or other graspable structure near the downstream end 110 of the embolic protection device 100 is connected to the retraction members 116, 120 by one or more connecting members 113. Optionally, two separate pull loops 122 may be provided for selectively retracting the upstream and downstream retraction members 116, 120. High strength magnets could be substituted for pull loops 122 (not shown) their opposite polarities being used to couple the device and a retraction apparatus or retraction member 116, 120. The retraction members 116, 120 and connecting members 113 may be made of suture, wire, plastic filament or a combination of these materials. In an alternate construction, the support hoops 112, 114 described above may also be configured to serve as the retraction members 116, 120.

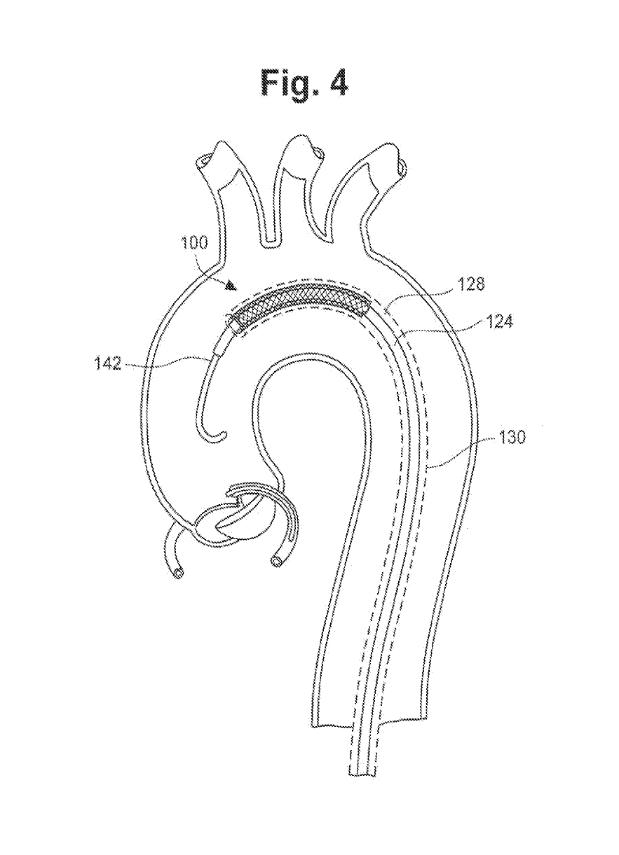

FIG. 4 shows an embolic protection device 100 in an undeployed condition mounted on a delivery catheter 126 being inserted over a guidewire 142 into a patient's aortic arch. Optionally, a delivery sheath 130 may be used to hold the embolic protection device 100 in the undeployed position. Once the embolic protection device 100 is at the desired location, the embolic protection device 100 is deployed, for example by withdrawing the delivery sheath 130 and allowing the embolic protection device 100 to expand. If the delivery catheter 126 is in the form of a diagnostic or therapeutic catheter, the catheter 126 can be advanced after the embolic protection device 100 is deployed to perform a diagnostic or interventional procedure. Optionally, the embolic protection device 100 can be retracted and withdrawn with the delivery catheter 126 after the diagnostic or interventional procedure has been completed. Alternatively, the delivery catheter 126 can be withdrawn, leaving the embolic protection device 100 in place.

FIG. 5 shows an embolic protection device 100 in a retracted condition for removal from the patient's aorta. A retrieval catheter 152 has been inserted intraluminally over a guidewire 146 to the location of the embolic protection device 100. Optionally, the guidewire 146 and retrieval catheter 152 may be inserted into the conical inner structure 104 and/or through the catheter port 106. A hook 154 on the distal end of an elongated member 156 within the retrieval catheter 152 has engaged the pull loop 122 on the embolic protection device 100. The hook 154 may engage the pull loop 122 through a distal port or a side port 158 on the retrieval catheter 152. The hook 154 and the pull loop 122 are withdrawn into the retrieval catheter 152, pulling on the connecting member 118 and causing the retraction members 116, 120 to tighten and collapse the embolic protection device 100 to a smaller diameter with the embolic debris 144 trapped inside the retracted embolic protection device 100.

The uninflated embolic protection device 100 may be delivered into the patient's aorta on a guidewire or delivery catheter and/or inside of a delivery sheath. Once, the embolic protection device 100 is in the proper position within the aortic arch, the inflatable support framework 160 is inflated through the inflation tube 170. At least the distal inflatable toroidal balloon 164, and optionally the proximal inflatable toroidal balloon 162, makes a seal with the aortic wall when inflated so that blood flow will be directed into the collection chamber 103 and through the filter mesh material to capture any potential emboli. If the embolic protection device 100 is intended for short term use, the proximal end of the inflation tube 170 may be left exposed at the insertion site. Alternatively, if the embolic protection device 100 is intended for long term use, the inflation tube 170 may be detached from the inflated embolic protection device 100. As another alternative, the proximal end of the inflation tube 170 may be buried under the patient's skin to allow later access for deflating and withdrawing the embolic protection device 100.

When the embolic protection device 100 is no longer needed, the inflatable support framework 160 is deflated and the embolic protection device 100 is withdrawn from the patient. Preferably, the embolic protection device 100 is configured such that the distal toroidal balloon 164 on the upstream end of the collection chamber 103 deflates first to effectively capture any potential emboli inside of the collection chamber 103. Other mechanisms described herein may also be used to assist in retracting the embolic protection device 100.

Other mechanisms may be employed for deploying and/or retrieving the embolic protection device 100. For example, the embolic protection device 100 can be elongated in the longitudinal direction to cause it contract radially. Releasing the tension on the embolic protection device 100 allows it to contract in the longitudinal direction and to expand radially for deployment. A retrieval catheter can be configured to apply longitudinal tension to the embolic protection device 100 to collapse it radially for withdrawal from the patient. Alternatively or in addition, the embolic protection device 100 can be twisted or wrapped to cause it contract radially. Releasing the embolic protection device 100 allows it to untwisted or unwrapped and to expand radially for deployment. A retrieval catheter can be configured to apply torque to the embolic protection device 100 to twist or wrap it to collapse it radially for withdrawal from the patient. These mechanisms may also be used in combination with the methods described above, such as those using retraction members or an inflatable support framework, to deploy and/or retrieve the embolic protection device 100.

Alternate embodiments of the embolic protection device 100 may combine features of the embodiments described herein to accomplish the same ends. For example, an embolic protection device 100 may be constructed with a single hoop 112 or inflatable toroidal balloon 164 on the upstream end of a cylindrical or conical outer structure 102 in contact with the vessel wall to anchor the device. The downstream end of the outer structure 102 may be constructed without a hoop or toroidal balloon, or alternatively with a smaller diameter hoop or toroidal balloon, as it is not critical for the downstream end of the embolic protection device 100 to contact or make a seal with the vessel wall. The embolic protection device of the present invention can also be used for embolic protection of other organ systems. For example, an embolic protection device can be deployed in the patient's descending aorta for preventing embolic particles in the aortic blood flow from entering the renal arteries and embolizing in the patient's kidneys.

The present invention, in one aspect, provides a high-flow intraluminal embolic protection device implantable in a blood vessel, the device comprising: a braided mesh-like tube of bio-compatible material having an expanded condition in which the tube diameter is larger than the diameter of the blood vessel in which it is to be implanted, the braided mesh-like tube having a length sufficient to be anchored to the source blood vessel, the braided mesh-like tube being dimensioned and configured to have in its implanted condition a porosity index such as to filter or capture antegrade-flowing emboli but not to unduly reduce the blood flow. The foregoing advantageous results have been found attainable when the braided mesh-like tube is designed to have, in its expanded condition, a porosity index of 55 to 80%, preferably 60-75%; windows or openings having an inscribed diameter of 30-480 microns, preferably 50-320 microns; and/or a diameter of wire filaments of 10-60 microns, preferably 20-40 microns; but when the filaments are of rectangular cross-section, a circumference 40-200 microns.

In the described preferred embodiments, the windows in the mesh-like tube produce a porosity index of preferably 60%-75%. The porosity index (P.I.) is defined by the relation:

$$P . I . = 1 - \frac{{S}_{m}}{{S}_{t}}$$

wherein: “Sₘ” is the actual surface covered by the mesh-like tube, and “Sₜ” is the total surface area of the mesh-like tube. In the tube devices of the present invention, however, the porosity index is not more than 80%, preferably 55-80%, more preferably 60-75%.

In the described preferred embodiments, the mesh-like tube includes windows having an inscribed diameter of 30-480 μm, preferably 50-320 μm, in the implanted condition of the mesh-like tube.

According to the described preferred embodiments, the mesh-like tube includes a plurality of filaments of bio-compatible material extending helically in an interlaced manner in opposite directions so as to form a braided tube. It is contemplated, however, that other mesh-like structures could be used, such as woven or knitted tubes.

A maximum porosity index is attained when the braiding angle, in the implanted condition of the braided tube, is 90°. Decreasing the implanted braiding angle below 90° increases the radial force applied by the braided tube against the inner surface of the blood vessel and decreases the P.I. Increasing the implanted braiding angle above 90° decreases the radial force applied by the braided tube against the inner surface of the blood vessel and decreases the P.I. In cases, where low radial force is needed, the desirable P.I. can thus be achieved by increasing the implanted braiding angle, as described below with respect to specific examples. Preferably, the braided tube has a braiding angle in the range of 20%-150% in the implanted condition of the braided tube.

Also in the described preferred embodiments, the filaments, or at least most of them, are of circular cross-section and have a diameter of 10-50 μm, preferably 20-40 μm. The filaments could also be of non-circular cross-section, such as of square or rectangular cross-section, in which case it is preferred that they have a circumference of 40-200 μm. It is also possible to use combination of several filament diameters and filament materials in one device to achieve structural stability and/or desired radio-opacity characteristic. Preferably the braid is formed of 24-144 filaments, more preferably 62-120 filaments. The filaments may be of a suitable bio-compatible material, metal or plastic, and may include a drug or other biological coating or cladding.

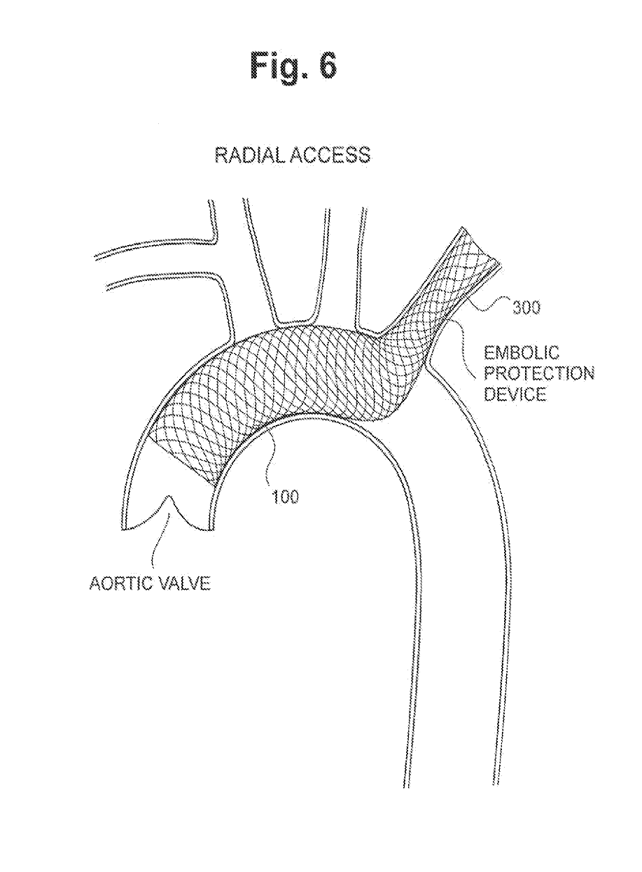

FIG. 6 shows schematically an embodiment of the present embolic protection device 100 partially deployed into the left subclavian artery 300.

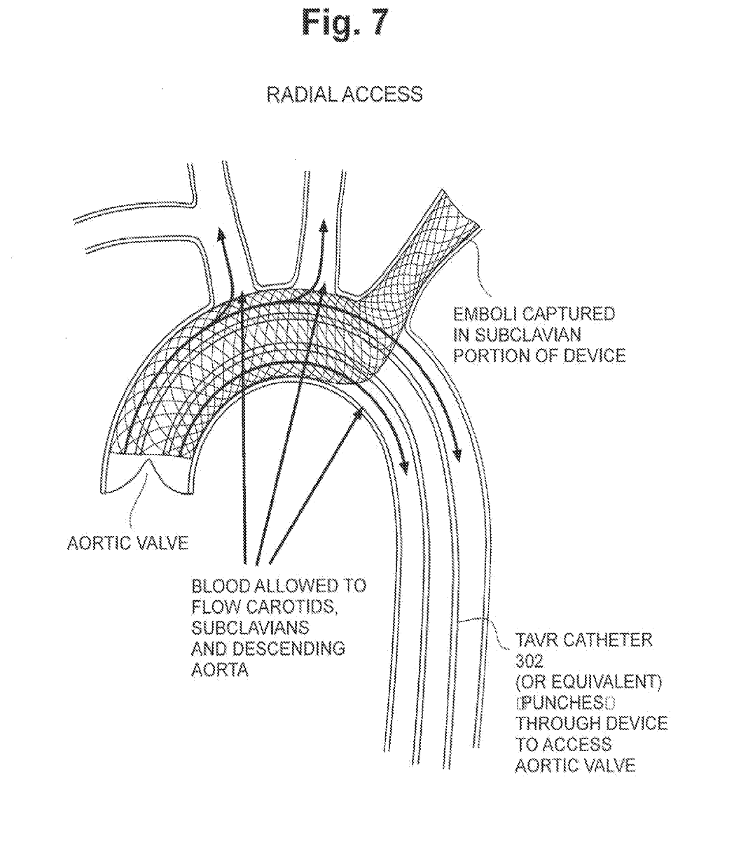

FIG. 7 shows the embodiment of FIG. 6 in which a TAVR catheter 302 accesses the aortic valve through the present embolic device.

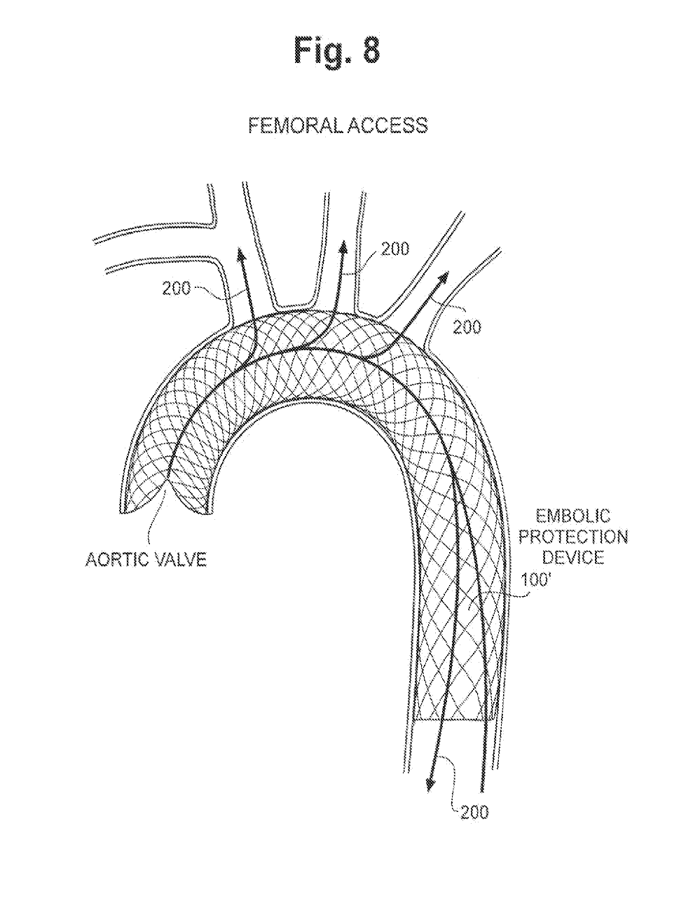

FIG. 8 shows deployment of a device of this invention 100′ into the descending aorta. The arrows 200 show possible directions of blood flow after emboli have been captured or filtered therefrom by the present device.

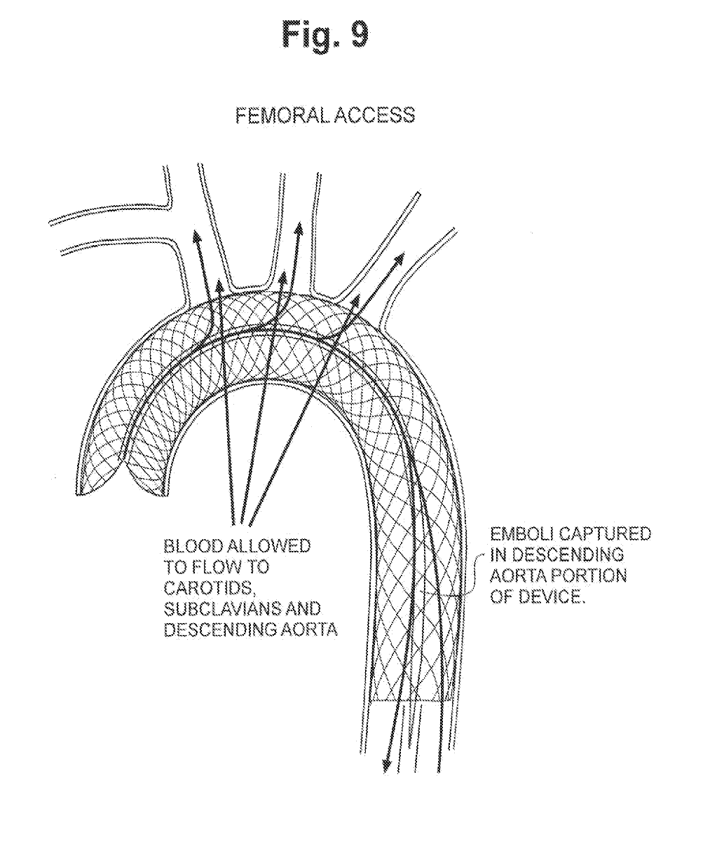

FIG. 9 shows the location in the descending aorta where emboli would be captured by the device shown in FIG. 8.

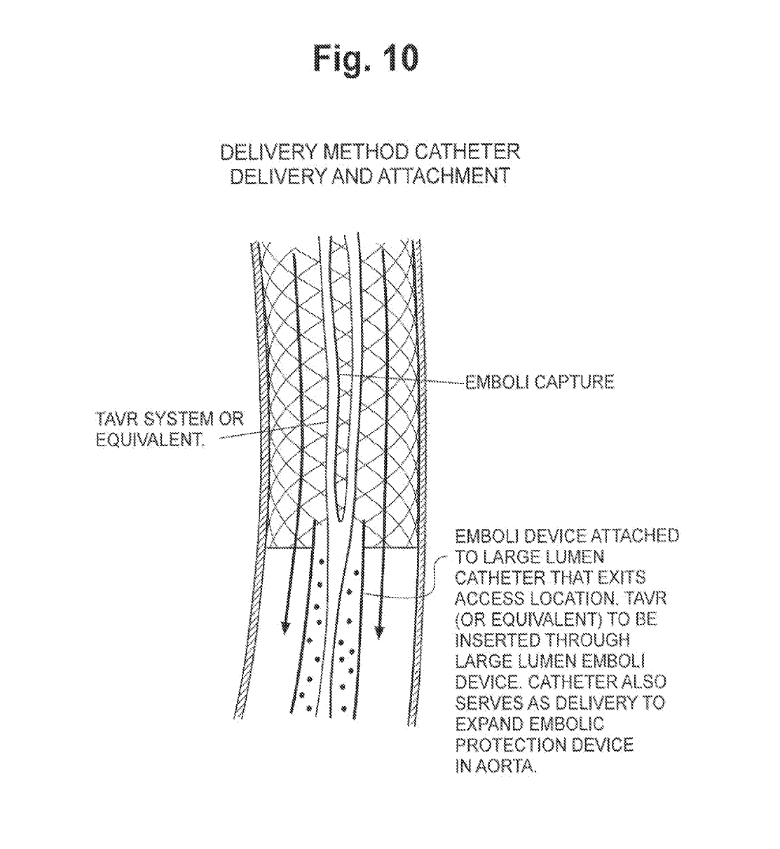

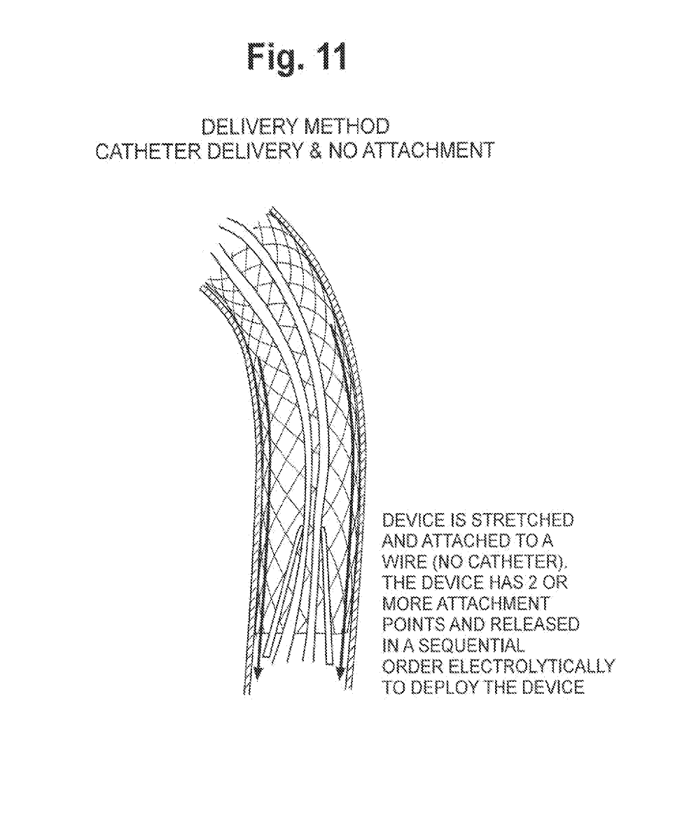

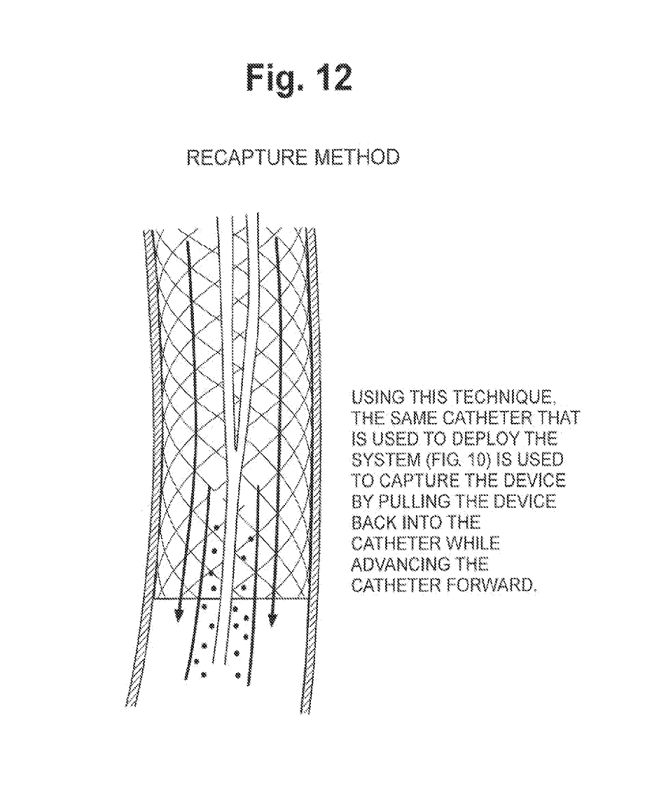

FIG. 10 illustrates withdrawal of device of this invention containing captured emboli using a large lumen catheter.

FIG. 11 shows the device of FIG. 10 with attachment points asserting embolic protection device withdrawal.

FIG. 12 shows capture of the emboli-containing device by withdrawal of same into the catheter.

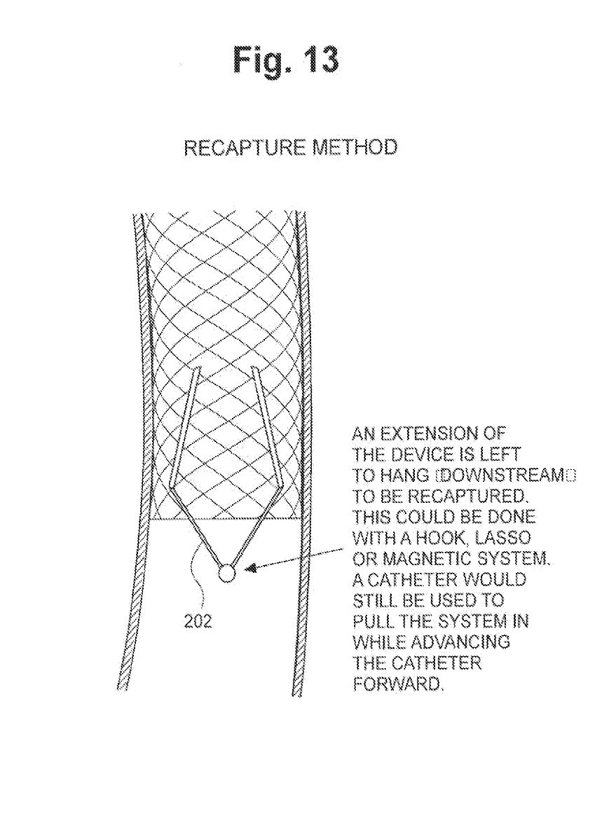

FIG. 13 illustrates a recapture hook 202 which can be used to assist device retrieval and withdrawal.

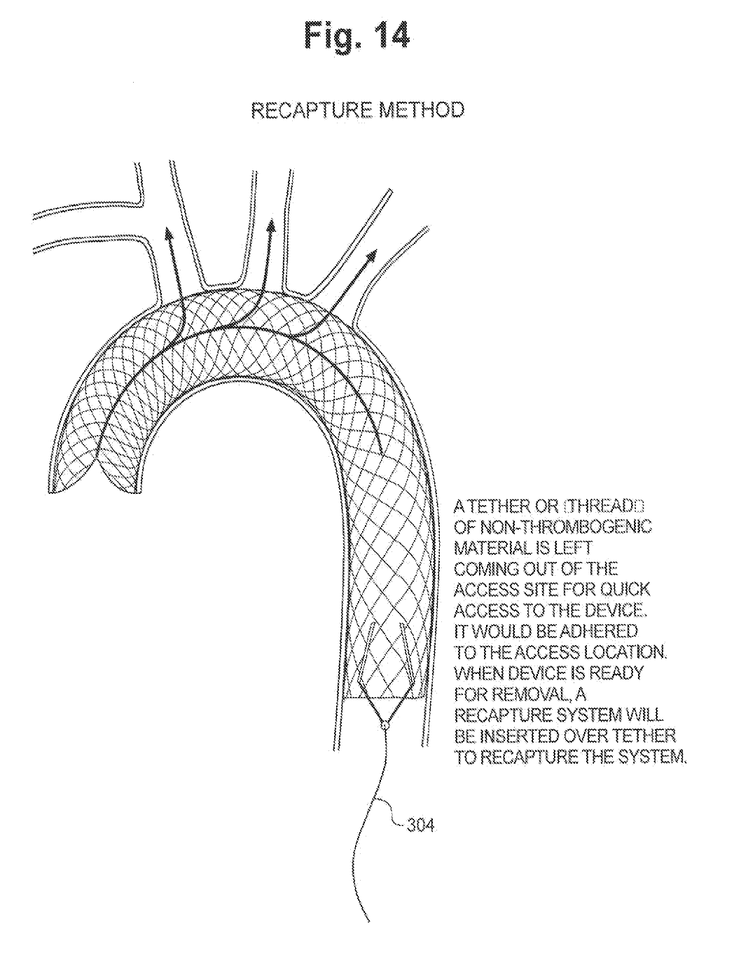

FIG. 14 illustrates and describes a further approach to embolic protection device withdrawal in which a thread is employed as a distally located (from the perspective of the patient) recapture mechanism.

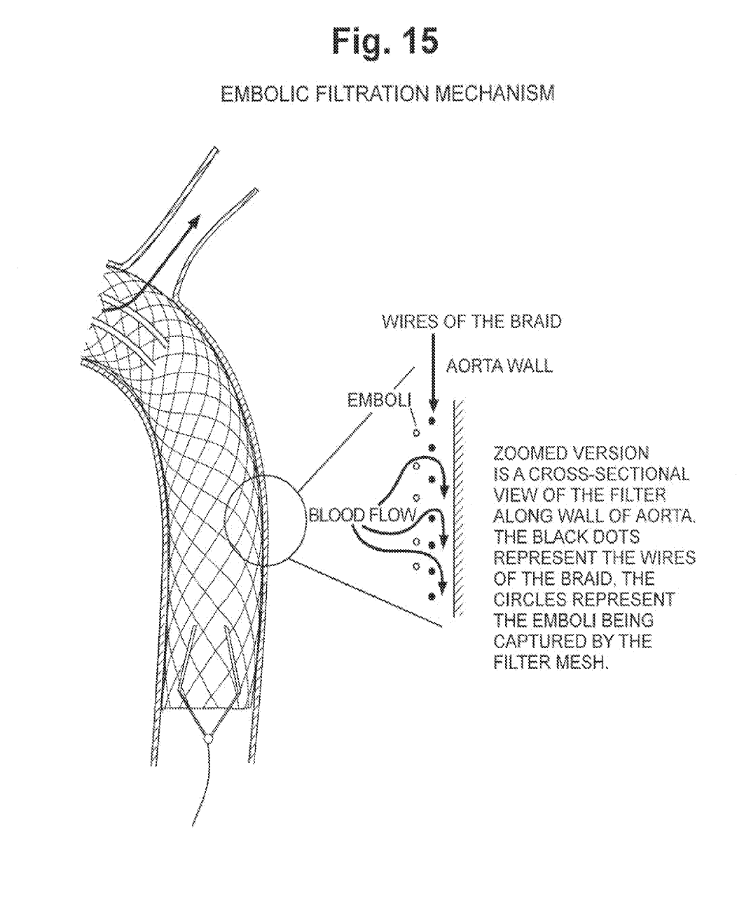

FIG. 15 is a detail sectional depiction of emboli being captured by an embolic protection device of the convention along the wall of the aorta.

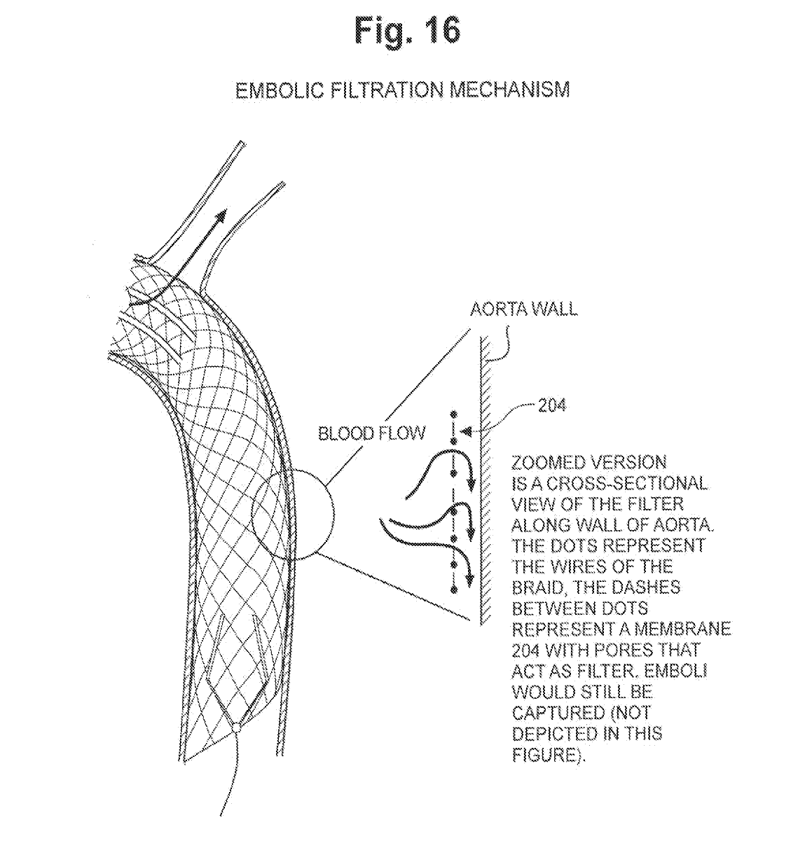

FIG. 16 shows an embodiment of the invention in which a porous membrane of polymer sieve or membrane 204 is deployed within, upon or outside the embolic protection device to capture emboli.

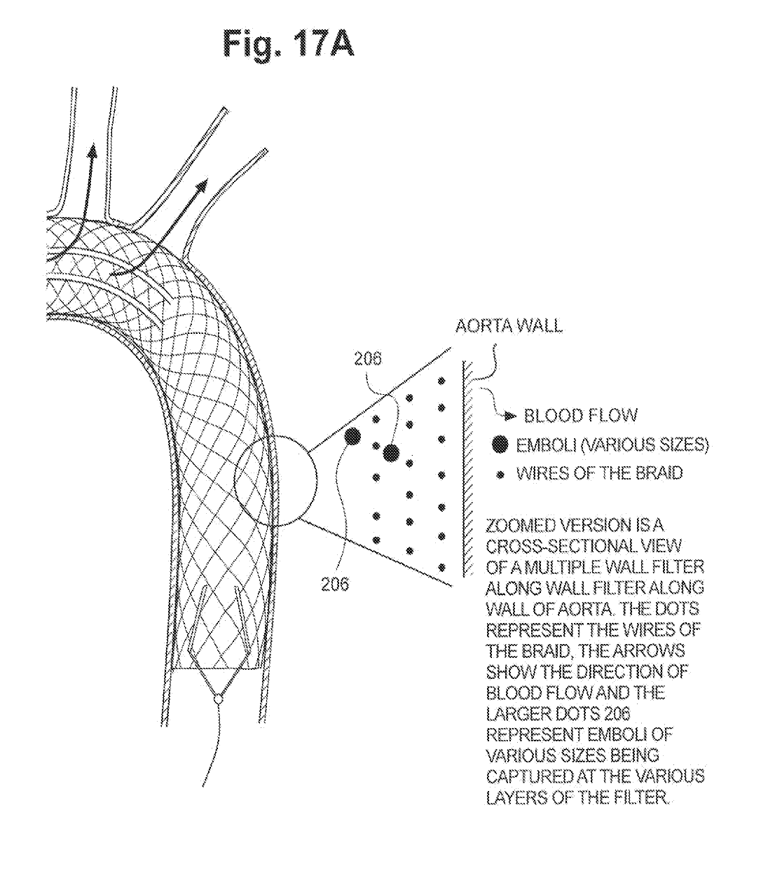

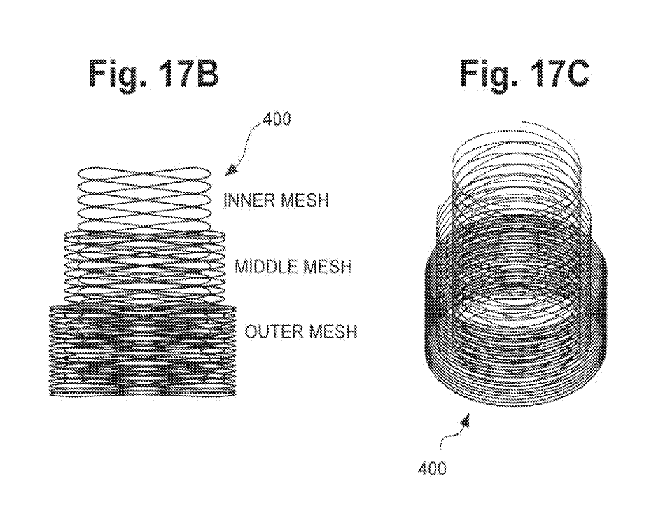

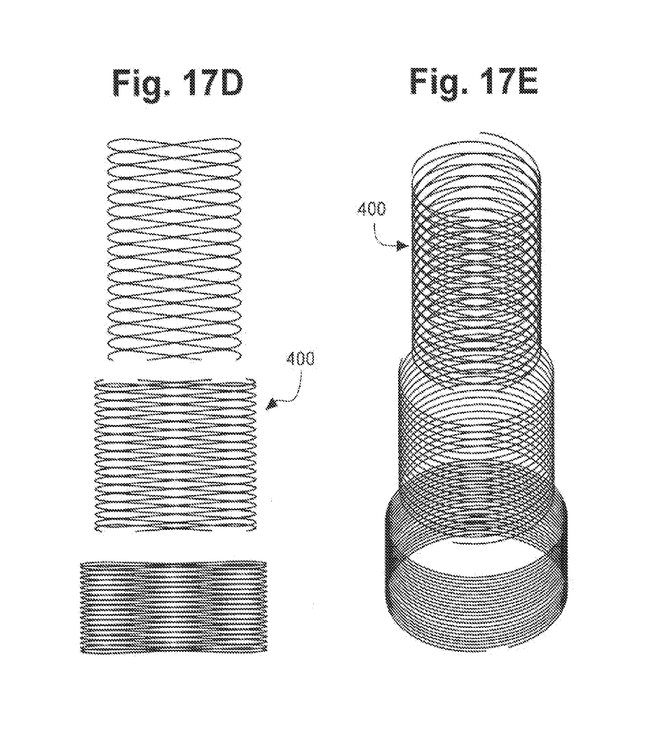

FIG. 17A shows a detailed schematic sectioned representation of emboli capture using a triaxial protection device of this invention. In this sectional view, a 3 coaxial 3 layer braided mesh-like embolic protection device is shown. Two (2) layer braided coaxial construction is also contemplated. Using a 3 coaxial 3 layer braided tube construction, the inner-most braided, mesh-like structure has the largest porosity, the middle tube has a smaller porosity and the outer-most braided tube is the least porous. The inner-most structure filters or traps the largest emboli, the middle coaxial braided tube structure filtering intermediate-sized emboli and the outer-most coaxial braided tube structure filtering the smallest emboli. That construction permits the maximum filtration of emboli from a high flow blood stream with minimal creation of vessel back pressure or resistance to flow.

FIGS. 17B and 17C show a side and perspective view of this triaxial braided mesh protection device 400. FIGS. 17D and 17E show partially assembled exploded views of device 400 prior to assembly.

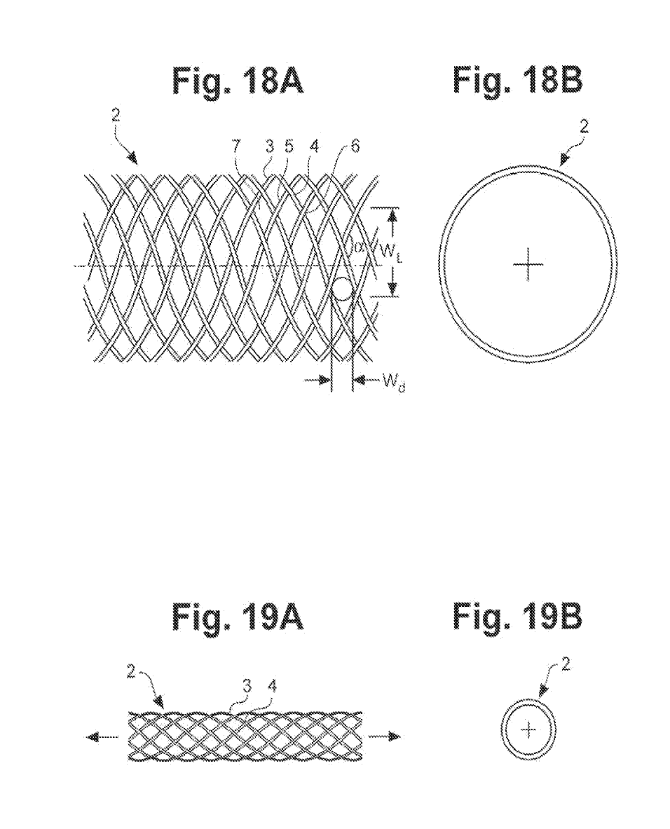

FIGS. 18a and 18b illustrate a detailed view of an intraluminal device, therein generally designated 2, constructed in accordance with the present invention in its implanted condition which it assumes in a blood vessel after deployment therein.

FIGS. 19a and 19b illustrate the intraluminal device 2 of FIGS. 18a and 18b in the contracted or stressed condition of the device which it assumes to facilitate its manipulation through the blood vessel to the deployment site.

As shown particularly in FIG. 18a, the intraluminal embolic protection device includes a plurality of filaments of elastic or non-elastic bio-compatible material, metal or plastic, extending helically in an interlaced manner to define a braided tube. Thus, shown in FIG. 18a is a first group of filaments 3 extending helically in one direction, and a second group of filaments 4 extending helically in the opposite direction, with the two groups of filaments being interwoven such that a filament 3 overlies a filament 4 at some points as shown at 5, and underlies a filament 4 at other points as shown at 6.

Filaments 3 and 4 thus define a braided woven tube having a plurality of windows 7. The inscribed diameter and the length of each window are shown at W_{d} and W_{L}, respectively, in the implanted condition of the braided tube. These characteristics depend on, among other factors including: the number of filaments; the cross section of the filaments; and the implanted angle “α” at the cross-over points of the two groups of filaments 3, 4. It is understood by those skilled in the art that the above dimensions describe the dimensions in the implanted condition of the braided tube. The dimensions in the fully expanded unimplanted condition will be somewhat different, with the angle “α” and W_{L} typically being larger than, and W_{d} typically being smaller than, the equivalent respective dimensions in the implanted state.

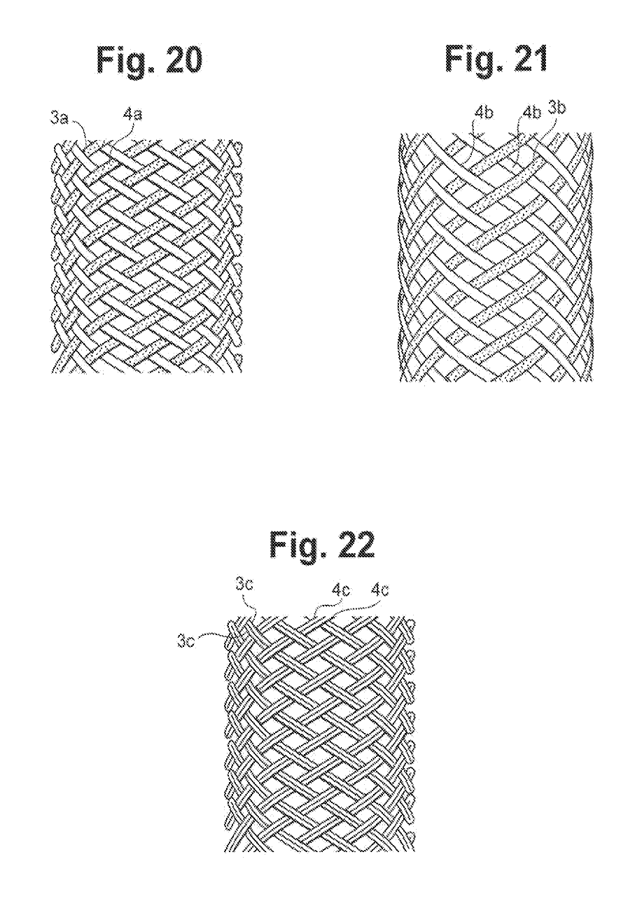

FIG. 20 more particularly illustrates the above-described braid pattern in the fully expanded condition of the braided tube. Thus, as shown in FIG. 20, each filament 3a extending helically in one direction is interwoven with one filament 4a extending helically in the opposite direction. Such a braid pattern is sometimes called a “one over one” pattern.

FIG. 21 illustrates a “one over two” pattern, in which each filament 3b extending helically in one direction is interwoven with two filaments 4b extending helically in the opposite direction.

FIG. 22 illustrates a further braid pattern that may be used, in which two (or more) contiguous filaments 3c extending helically in one direction are interwoven with two (or more) contiguous filaments 4c extending helically in the opposite direction.

The braid pattern illustrated in FIG. 20 is of highest flexibility, whereas that illustrated in FIG. 22 is of lower flexibility but of higher strength.

Braided-tube intraluminal devices are used in other systems, for example as described in Wallsten et al, U.S. Pat. No. 5,061,275 and Wallsten U.S. Pat. No. 4,954,126, the contents of which are incorporated herein by reference. They are generally used as stents for providing support to a wall of a blood vessel, for implanting a graft, e.g., to treat an aneurysm (FIG. 9 of the latter patent), or for other purposes. In other contexts, the braided tube sometimes is shown to have an expanded, unimplanted condition having a diameter slightly larger than the diameter of the intended blood vessel in which it is to be implanted so that when the device is deployed it becomes firmly embedded in the wall of blood vessel. The braided tube is capable of being stressed into a contracted condition, as shown in FIGS. 19a and 19b, wherein the diameter of the braided tube is decreased, and its length increased, to permit manipulation of the braided tube through the blood vessel to the site of implantation.

Further information concerning the construction and deployment of such braided-tube intraluminal devices is available in the above-cited patents, and also in U.S. patent application Ser. No. 10/311,876, filed on Dec. 20, 2002, entitled “Implantable Braided Stroke Preventing Device and Method of Manufacturing”, the contents of which are incorporated herein by reference.

According to the present invention, the constituent element making up the mesh-like tube are of a sufficiently small size in cross-section and define windows of a size such that the mesh-like tube, when in its contracted condition, is sufficiently flexible so as to be easily manipulatable through the blood vessel to be implanted in e.g., an artery; and when in its implanted condition anchoring itself to both the source blood vessel/artery and filtering/capturing emboli flowing therethrough. The skewing is caused by the flow of blood through the walls of the mesh-like tube, and the amount of skew is a function of the predetermined implanted porosity index. In an exemplary embodiment, in which the mesh-like tube is constituted of braided filaments, the windows defined by the filaments of the braided tube are such as to filter emboli from the blood, but does not unduly reduce the blood flow to the branch vessels to the degree likely to cause damage to tissues supplied with blood by such vessels.

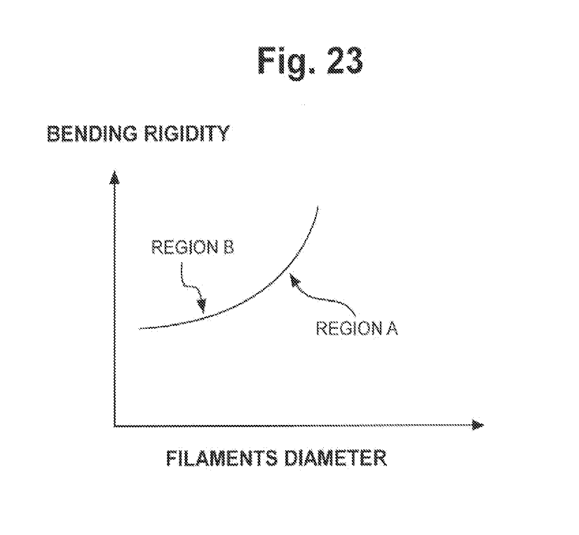

FIG. 23 schematically illustrates how the bending rigidity or flexibility of a braided tube varies with the diameter of the filaments. Region A in FIG. 23 illustrates typical diameters in conventional stents used for supporting blood vessels, which region usually starts above 60 μm and extends to several hundred μm. Region B in FIG. 23 illustrates the region of filament diameters for use in constructing braided tubes in accordance with the present invention. The filament diameters in this region would be significantly smaller than in region A, preferably being 10-50 μm, more preferably 20-40 μm.

The foregoing dimensions apply to the diameters of filaments of circular cross-section. Where the filaments are of non-circular cross-section, such as of rectangular or square cross-section, the filaments would preferably have a circumference of 40-200 μm. The circumference is defined in macro scale. The circumference can be enlarged at the micro-scale level by adding roughness to the wire, in order to control the neointimal growth and making the circumference in micro scale longer while keeping the macro scale the same. In this case the surface cross section of the filament would be in the range 75-3000 μm^{̂2} preferably 300-1300μ^{̂2}.

As indicated earlier, the windows formed in the braided mesh-like tube would also be preferably within a predetermined range such as to filter the blood-flow, but maintain sufficient blood flow in or to the branch vessels. Preferably the length of the window, i.e., its long dimension as shown at W_{L} in FIG. 18a, would be within the range of 30-480 μm, more preferably 50-320 μm, in the implanted condition of the braided tube. Also, the implanted angle (α, FIG. 18a) would preferably be within the range of 20°-150°, more preferably 40-80° for high radial force and 100-140° for low radial force, in the implanted condition of the braided tube. In yet another preferred embodiment the braid angle in the implanted condition is approximately 90°, preferably in the range of 70°-110°. The diameter and length of the braided tube in its normal, implanted condition will vary according to the location and anatomical dimensions at the particular site of the implantation. Preferably, the windows are preferably globally (but not necessary locally) uniform in size.

The filaments of the exemplary braided mesh-like tube embodiment can be made of any suitable material which are bio-compatible and which can be worked into a braid. Bio-compatible herein includes any material that can be safely introduced and implanted in human or animal bodies for indefinite periods of time without causing any significant physiological damage. Preferably, the filaments are made of a material selected from among the 316L stainless steel, tantalum, and super elastic Nitinol, cobalt base alloy, polymer or any other suitable metal or metal combination.

Filaments also can be coated with bio-compatible coatings [Ulrich Sigwart, “Endoluminal Stenting”, W. B. Saunders Company Ltd., London, 1996]. It is possible to use a combination of several filament materials in one device and combinations of several materials in one filament. The above embodiments have been described in relation to a braided mesh-like tube, however this is not meant to be limiting in any way. Other mesh-like structures, such as woven or knitted tubes exhibiting similar porosity and flexibility can be used without exceeding the scope of the invention.

In some situations, it may be desired to implant the device in a portion of a lumen, e.g., an artery, varying significantly in diameter along its length. As will be appreciated, if a constant diameter braided tube device is inserted into such a variable-diameter lumen, this may result in a defective anchoring of the device at the larger diameter portion of the lumen, and in a possible risk of the migration of the device within the lumen. This problem can be easily overcome in several ways, e.g., by creating braided devices with variable diameters along their longitudinal axis, or varying the pitch along the longitudinal axis, as described in the above-cited U.S. patent application Ser. No. 10/311,876 is incorporated herein by reference.

U.S. Pat. No. 8,414,482 to Belson and U.S. Pat. No. 7,942,921 to Yodfat et al. are specifically incorporated herein in their entireties.

While the present invention has been described herein with respect to the exemplary embodiments and the best mode for practicing the invention, it will be apparent to one of ordinary skill in the art that many modifications, improvements and subcombinations of the various embodiments, adaptations and variations can be made to the invention without departing from the spirit and scope thereof.

What is claimed:

1. An embolic protection device, comprising:
a filter comprising a braided mesh-like tube defining a collection chamber for captured emboli, the filter having a deployed condition wherein an outer periphery of the filter contacts a blood vessel wall to direct blood flow and filters antegrade-flowing potential emboli from the blood flow and direct them into the collection chamber.

2. The embolic protection device of claim 1, a catheter port which permits the passage of a catheter into the filter.

3. An embolic protection device, comprising:
an approximately cylindrical outer structure made of a braided, mesh-like material wherein;
an upstream end of the embolic protection device is open for blood to flow into the cylindrical outer structure which defines collection chamber for captured emboli.

4. The embolic protection device of claim 3, wherein the braided mesh-like material of the cylindrical outer structure is made from a mesh-like fabric of knitted or woven filaments or wires and with a pore size chosen to prevent emboli over a predetermined size from passing therethrough.

5. The embolic protection device of claim 3, wherein the woven mesh-like material of the cylindrical outer structure is made of a metal, a polymer or a combination thereof.

6. The embolic protection device of claim 3, wherein the woven mesh-like material of the cylindrical outer structure has an antithrombogenic coating on its surface.

7. The embolic protection device of claim 3, wherein the woven mesh-like material of the cylindrical outer structure has a pore size in the range of approximately 1 mm to 0.1 mm.

8. The embolic protection device of claim 3, wherein the embolic protection device has an undeployed retracted condition and a deployed expanded condition.

9. The embolic protection device of claim 8, further including a delivery catheter configured to deliver the embolic protection device into a blood vessel of a patient in the undeployed, retracted condition.

10. The embolic protection device of claim 9, further comprising a tubular outer delivery sheath to maintain the embolic protection device in the undeployed retracted condition prior to deployment.

11. The embolic protection device of claim 8, wherein the woven mesh-like material of the cylindrical outer structure is a resilient material that can be compressed into the undeployed retracted condition and that will self-expand into the deployed expanded condition.

12. The embolic protection device of claim 3, further comprising at least one retraction member encircling the circumference of the cylindrical outer structure.

13. The embolic protection device of claim 12, further comprising a pull loop or other graspable structure near the downstream end of the embolic protection device connected to the retraction members.

14. The embolic protection device of claim 3, wherein the embolic protection device is retractable for retrieval from a patient's blood vessel.

15. An intraluminal emboli collection device implantable in a blood vessel comprising: a braided mesh-like tube of bio-compatible material having an expanded condition in which the tube diameter is larger than the diameter of the blood vessel in which it is to be implanted, and having a length sufficient to be anchored at both ends to said blood vessel; said braided mesh-like tube being constituted of 24-144 filaments, and being designed to have in its implanted condition a porosity index of 60-75% and windows having an inscribed diameter of 50-320 μm.

16. The intraluminal device according to claim 15, wherein said braided mesh-like tube is constituted of multiple tubular meshes, lying one above the other in layer-like formations, in the implanted condition of the braided mesh-like tube.

17. A method of treating an aneurysm in a blood vessel, comprising: implanting in the blood vessel a braided mesh-like tube of bio-compatible material having an expanded condition in which the tube diameter is larger than the diameter of the blood vessel in which it is to be implanted, and having a length sufficient to be anchored at both ends to said blood vessel; said braided mesh-like tube being constituted of 24-144 filaments, and being designed to have in its implanted condition a porosity index of 60-75% and windows having an inscribed diameter of 50-320 μm.

18. The method according to claim 17, wherein each of said filaments has a circular cross-section has a diameter of 10-50 μm.

19. The method according to claim 17, wherein said braided mesh-like tube is formed of 62-120 filaments of bio-compatible material.

20. The method according to claim 17, wherein said braided mesh-like tube is constituted of a single tubular mesh.

21. The method according to claim 17, wherein said braided mesh-like tube is constituted of multiple tubular meshes, lying one above the other in layer-like formations, in the implanted condition of the braided mesh-like tube.

22. An assembly for implanting an intraluminal device in a selected site of a blood vessel, the assembly comprising: an intraluminal device, which comprises a braided mesh-like tube of bio-compatible material having an expanded condition in which the tube diameter is larger than the diameter of the blood vessel in which it is to be implanted, and having a length sufficient to be anchored at both ends to said blood vessel; said braided mesh-like tube being constituted of 24-144 filaments, and being designed to have in its implanted condition a porosity index of 60-75% and windows having an inscribed diameter of 50-320 μm, and a microcatheter for delivering said intraluminal device to said selected site in the blood vessel and for implanting it therein.

23. The assembly according to claim 22, wherein each of said filaments has a circular cross-section having a diameter of 10-50 μm.

24. The assembly according to claim 22, wherein said braided mesh-like tube is formed of 62-120 filaments of bio-compatible material.

25. The assembly according to claim 22, wherein said braided mesh-like tube is constituted of a single tubular mesh.

26. The assembly according to claim 22, wherein said braided mesh-like tube is constituted of multiple tubular meshes, lying one above the other in coaxial layer-like formations, in the implanted condition of the braided mesh-like tube.
